(19) United States
(12) Patent Application Publication
(10) Pub. No.: US 2024/0383909 A1
(43) Pub. Date: Nov. 21, 2024

(54) CO-CRYSTALS

(71) Applicant: INTRA-CELLULAR THERAPIES, INC., New York, NY (US)

(72) Inventor: Peng LI, New Milford, NJ (US)

(21) Appl. No.: 18/688,618

(22) PCT Filed: Sep. 2, 2022
(86) PCT No.: PCT/US2022/075902

§ 371 Date: Mar. 1, 2024

Related U.S. Application Data
(60) Provisional application No. 63/260,873, filed on Sep. 3, 2021.

(51) Int. Cl. C07D 487/14; C07C 39/08; C07C 215/12; C07D 209/48

(52) U.S. Cl. CPC C07D 487/14; C07C 39/08; C07C 215/12; C07D 209/48

(57) ABSTRACT

The present disclosure relates to co-crystals of (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one, compositions comprising the same, as well as methods of making and using such co-crystals.

### FIELD OF THE DISCLOSURE

The present disclosure relates to co-crystals of (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one, compositions comprising the same, as well as methods of making and using such co-crystals.

### BACKGROUND OF THE DISCLOSURE

The compound (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one is disclosed in WO 2009/075784 (U.S. Pub. No. 2010/0273754). This compound has been found to be a potent and selective phosphodiesterase 1 (PDE 1) inhibitor useful for the treatment or prophylaxis of disorders characterized by low levels of cAMP and/or cGMP in cells expressing PDE1, and/or reduced dopamine D1 receptor signalling activity (e.g., neurodegenerative disorders such as Parkinson's disease; cognitive impairment of schizophrenia; cardiovascular disorders such as cardiac hypertrophy, heart failure and hypertension; cancers such as gliomas and leukemia; and renal disorders such as kidney disease); and/or any disease or condition that may be ameliorated by the enhancement of progesterone signalling. This list of disorders is exemplary and not intended to be exhaustive.

The publication WO 2009/075784 discloses (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one in free base form and generally in pharmaceutically acceptable salt form, but no specific salt was shown to have particular stability or desired properties. Because many pharmaceutical compounds can exist in different physical forms (e.g., liquid or solid in different crystalline, amorphous, polymorphous, hydrate or solvate forms) which can vary the stability, solubility, bioavailability or pharmacokinetics (absorption, distribution, metabolism, excretion or the like) and/or bioequivalence of a drug, it is of critical importance in the pharmaceutical development to identify a pharmaceutical compound of optimal physical form (e.g., free base or salt in solid, liquid, crystalline, hydrate, solvate, amorphous or polymorphous forms).

### SUMMARY OF THE INVENTION

In a first aspect, the present disclosure is directed to a co-crystal [Co-crystal 1] comprising:
A) (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) in free, pharmaceutically acceptable salt or prodrug form, including its enantiomers, diastereoisomers and racemates; and
B) a co-crystal former.

Therefore, in the first aspect, the disclosure provides the following:
1.1 Co-crystal 1, wherein Compound 1 is in free base or salt form.
1.2 Co-crystal 1 or 1.1, wherein Compound 1 is in free base form.
1.3 Any of the preceding Co-crystals, wherein the co-crystal is anhydrous or solvated.
1.4 Any of the preceding Co-crystals, wherein the co-crystal is anhydrous.
1.5 Any of Co-crystals 1.1-1.3, wherein the co-crystal is solvated.
1.6 Any of the preceding Co-crystals, wherein the ratio of Compound 1 to co-crystal former is between 1:0.5 and 1:3 (e.g., 1:0.5, 1:0.7, 1:1 or 1:2).
1.7 Any of the preceding Co-crystals, wherein the co-crystal former is selected from alanine, glutamic acid, 2-aminobutyric acid, urea, tyrosine, glycine, arginine, 6-hydroxy nicotinamide, diethanolamine, 3-Nitro phthalimide, isoleucine, histidine, bis acetyled ethylenediamide, nicotinamide, acetanilide, leucine, lysine, isonicotinamide, resorcinol, 4-nitro phthalimide, proline, serine, pyridino phthalimide, 4-acetamidophenol Benzamide, valine, threonine, tromethamine, hydroquinone, carbamazepine, phenylalanine, cysteine, 3-aminobutyric acid, piperazine, 4-acetamidophenol, tryptophan, methionine, 6-methylpyridine-3-carboxamide, succinimide, aspartic acid, asparagine, mono acetyled ethylenediamine, 4-methylacetanilide, glutamine, 2-pyridone, pyromellitic diimide, 1,2-dihydroxybenzene, amino-4,6-dimethyl nicotinamide, and salts thereof.
1.8 Any of the preceding Co-crystals, wherein the co-crystal former is selected from tyrosine, 3-nitro-phthalimide, pyridino phthalimide, diethanolamine, resorcinol, hydroquinone, threonine, and salts thereof.
1.9 Any of the preceding Co-crystals, wherein the co-crystal former is selected from 3-nitro-phthalimide, diethanolamine, resorcinol, hydroquinone, and salts thereof.
1.10 Any of the preceding Co-crystals, wherein the co-crystal former is 3-nitro-phthalimide.
1.11 Co-crystal 1.10, wherein the co-crystal exhibits an X-ray powder diffraction pattern comprising at least five peaks having 2-theta angle values selected from the group consisting of: 7.8, 16.3, 17.0, 17.4, 18.6, 19.1, 19.6, 20.8, 21.4, and 21.5 degrees, wherein the XRPD pattern is measured in a diffractometer using copper anode, e.g., at wavelength alpha1 of 1.5406 Å and wavelength alpha2 of 1.5444 Å.
1.12 Any of Co-crystals 1.10 or 1.11, wherein the co-crystal exhibits an X-ray powder diffraction pattern comprising peaks having 2-theta angle values at: 16.3, 17.0, 17.4, 18.6, and 19.1 degrees, wherein the XRPD pattern is measured in a diffractometer using copper anode, e.g., at wavelength alpha1 of 1.5406 Å and wavelength alpha2 of 1.5444 Å.
1.13 Any of Co-crystals 1.10-1.12, wherein the co-crystal exhibits an X-ray powder diffraction pattern comprising at least five peaks having 2-theta angle values selected from those set forth in the table below:

| | | | | Rel. |
|---|---|---|---|---|
| Index | Angle | d Value | Intensity | Intensity |
| 1 | 7.807 | 11.31578 | 467 | 14.50% |
| 2 | 10.695 | 8.2655 | 173 | 5.40% |
| 3 | 16.256 | 5.4483 | 1010 | 31.50% |
| 4 | 16.989 | 5.2148 | 953 | 29.60% |
| 5 | 17.403 | 5.09173 | 2210 | 68.80% |
| 6 | 17.817 | 4.97425 | 341 | 10.60% |
| 7 | 18.571 | 4.77403 | 1110 | 34.50% |
| 8 | 19.052 | 4.65455 | 3220 | 100.00% |
| 9 | 19.637 | 4.51712 | 743 | 23.10% |
| 10 | 20.806 | 4.26583 | 838 | 26.00% |
| 11 | 21.026 | 4.22184 | 379 | 11.80% |
| 12 | 21.431 | 4.14283 | 929 | 28.90% |
| 13 | 21.531 | 4.12395 | 821 | 25.50% |
| 14 | 23.171 | 3.83555 | 274 | 8.50% |
| 15 | 24.355 | 3.65167 | 397 | 12.30% |

wherein the XRPD pattern is measured in a diffractometer using copper anode, at wavelength alpha1 of 1.5406 Å and wavelength alpha2 of 1.5444 Å.
1.14 Any of Co-crystals 1.10-1.13, wherein said co-crystal exhibits an X-ray powder diffraction pattern comprising at least five peaks having d-spacing values selected from those set forth in the table of Co-crystal 1.13.
1.15 Any of Co-crystals 1.10-1.14, wherein the co-crystal exhibits an X-ray powder diffraction pattern comprising at least five peaks having d-spacing values selected from the group consisting of 11.32, 5.45, 5.21, 5.09, 4.77, 4.65, 4.52, 4.27, 4.14, and 4.12 Å.
1.16 Any of Co-crystals 1.10-1.15, wherein the co-crystal exhibits an X-ray powder diffraction pattern comprising peaks having d-spacing values of 5.45, 5.21, 5.09, 4.77, and 4.65 Å.
1.17 Any of Co-crystals 1.10-1.16, wherein said co-crystal exhibits an X-ray powder diffraction pattern corresponding with or substantially as depicted in the table of Co-crystal 1.13 or in FIG. 1.
1.18 Any of Co-crystals 1.10-1.17, wherein said co-crystal exhibits a Differential Scanning calorimetry (DSC) pattern comprising an endothermic peak at about 110° C.-111∪ C., e.g., about 110.5° C.
1.19 Any of Co-crystals 1.10-1.18, wherein the co-crystal exhibits a Differential Scanning calorimetry (DSC) pattern corresponding with or substantially as depicted in FIG. 2.
1.20 Any of Co-Crystals 1.10-1.19, wherein said co-crystal exhibits a Differential Scanning calorimetry (DSC) pattern comprising an endothermic peak at about 86° C.-87° C., e.g., about 86.5° C.
1.21 Any of Co-crystals 1.10-1.20, wherein the co-crystal exhibits a Differential Scanning calorimetry (DSC) pattern corresponding with or substantially as depicted in FIG. 3.
1.22 Any of Co-crystals 1.10-1.21, wherein the co-crystal has a needle shape, e.g., having a needle shape with a length of 50-200 μm.
1.23 Any of Co-crystals 1.10-1.22, wherein the co-crystal comprises Compound 1 in free base form and co-crystal former in a ratio of 1:0.5 and/or 1:1 (e.g., 1:0.7).
1.24 Any of Co-crystals 1.1-1.9, wherein the co-crystal former is diethanolamine.
1.25 Co-crystal 1.24, wherein the co-crystal exhibits an X-ray powder diffraction pattern comprising at least five peaks having 2-theta angle values selected from the group consisting of: 7.29, 7.70, 7.80, 9.987, 13.78, 16.59, 16.93, 18.84, 20.66, and 20.68 degrees, wherein the XRPD pattern is measured in a diffractometer using copper anode, e.g., at wavelength alpha1 of 1.5406 Å and wavelength alpha2 of 1.5444 Å.
1.26 Any of Co-crystals 1.24-1.25, wherein the co-crystal exhibits an X-ray powder diffraction pattern comprising peaks having 2-theta angle values at 7.29, 7.70, 7.80, 13.78, and 18.84 degrees, wherein the XRPD pattern is measured in a diffractometer using copper anode, e.g., at wavelength alpha1 of 1.5406 Å and wavelength alpha2 of 1.5444 Å.
1.27 Any of Co-crystals 1.24-1.26, wherein the co-crystal exhibits an X-ray powder diffraction pattern comprising at least five peaks having 2-theta angle values selected from those set forth in the table below:

| | | | | Rel. |
|---|---|---|---|---|
| Index | Angle | d Value | Intensity | Intensity |
| 1 | 4.636 | 19.04449 | 479 | 32.50% |
| 2 | 7.286 | 12.1232 | 1250 | 84.90% |
| 3 | 7.702 | 11.46886 | 1470 | 100.00% |
| 4 | 7.795 | 11.33322 | 1030 | 70.00% |
| 5 | 9.196 | 9.60955 | 314 | 21.30% |
| 6 | 9.987 | 8.85011 | 604 | 40.90% |
| 7 | 13.486 | 6.56056 | 433 | 29.30% |
| 8 | 13.773 | 6.42438 | 810 | 54.90% |
| 9 | 14.123 | 6.26599 | 285 | 19.30% |
| 10 | 14.63 | 6.05003 | 199 | 13.50% |
| 11 | 16.587 | 5.34025 | 625 | 42.40% |
| 12 | 16.931 | 5.23254 | 635 | 43.00% |
| 13 | 17.904 | 4.95021 | 474 | 32.10% |
| 14 | 18.841 | 4.70617 | 910 | 61.70% |
| 15 | 20.39 | 4.35202 | 381 | 25.90% |
| 16 | 20.678 | 4.29208 | 730 | 49.50% |
| 17 | 20.66 | 4.2957 | 693 | 47.00% |
| 18 | 21.338 | 4.16067 | 539 | 36.60% |
| 19 | 23.108 | 3.84586 | 343 | 23.30% |
| 20 | 26.631 | 3.34459 | 281 | 19.00% |

wherein the XRPD pattern is measured in a diffractometer using copper anode, at wavelength alpha1 of 1.5406 Å and wavelength alpha2 of 1.5444 Å.
1.28 Any of Co-crystals 1.24-1.27, wherein said co-crystal exhibits an X-ray powder diffraction pattern comprising at least five peaks having d-spacing values selected from those set forth in the table of Co-crystal 1.27.
1.29 Any of Co-crystals 1.24-1.28, wherein the co-crystal exhibits an X-ray powder diffraction pattern comprising at least five peaks having d-spacing values selected from the group consisting of 12.12, 11.49, 11.33, 8.85, 6.42, 5.34, 5.23, 4.71, 4.30, and 4.29 Å.
1.30 Any of Co-crystals 1.24-1.29, wherein the co-crystal exhibits an X-ray powder diffraction pattern comprising peaks having d-spacing values of 12.12, 11.49, 11.33, 6.42, and 4.71 Å.
1.31 Any of Co-crystals 1.24-1.30, wherein said co-crystal exhibits an X-ray powder diffraction pattern corresponding with or substantially as depicted in the table of Co-crystal 1.24 or in FIG. 4.
1.32 Any of Co-crystals 1.24-1.31, wherein said co-crystal exhibits a Differential Scanning calorimetry (DSC) pattern comprising an endothermic peak at about 121° C.-122° C., e.g., about 121.7° C.
1.33 Any of Co-crystals 1.24-1.32, wherein the co-crystal exhibits a Differential Scanning calorimetry (DSC) pattern corresponding with or substantially as depicted in FIG. 5.
1.34 Any of Co-crystals 1.24-1.33, wherein the co-crystal has a needle shape, e.g., having a needle shape with a length of 5-40 μm.
1.35 Any of Co-crystals 1.24-1.34, wherein the co-crystal comprises Compound 1 in free base form and co-crystal former in a ratio of 1:1.
1.36 Any of Co-crystals 1.1-1.9, wherein the co-crystal former is resorcinol.
1.37 Co-crystal 1.36, wherein the co-crystal exhibits an X-ray powder diffraction pattern comprising at least five peaks having 2-theta angle values selected from the group consisting of: 7.3, 7.4, 10.0, 14.1, 17.0, 18.7, 19.0, 19.7, 22.9, and 23.7 degrees, wherein the XRPD pattern is measured in a diffractometer using copper anode, e.g., at wavelength alpha1 of 1.5406 Å and wavelength alpha2 of 1.5444 Å.
1.38 Any of Co-crystals 1.36-1.37, wherein the co-crystal exhibits an X-ray powder diffraction pattern comprising peaks having 2-theta angle values at 7.3, 7.4, 17.0, 19.0, and 22.9 degrees, wherein the XRPD pattern is measured in a diffractometer using copper anode, e.g., at wavelength alpha1 of 1.5406 Å and wavelength alpha2 of 1.5444 Å.
1.39 Any of Co-crystals 1.36-1.38, wherein the co-crystal exhibits an X-ray powder diffraction pattern comprising at least five peaks having 2-theta angle values selected from those set forth in the table below:

| | | | | Rel. |
|---|---|---|---|---|
| Index | Angle | d Value | Intensity | Intensity |
| 1 | 7.276 | 12.13973 | 1760 | 98.40% |
| 2 | 7.36 | 12.00098 | 1790 | 100.00% |
| 3 | 9.952 | 8.88113 | 737 | 41.20% |
| 4 | 10.146 | 8.7117 | 465 | 26.00% |
| 5 | 14.118 | 6.26829 | 511 | 28.60% |
| 6 | 14.649 | 6.04204 | 397 | 22.20% |
| 7 | 15.962 | 5.54788 | 478 | 26.70% |
| 8 | 16.362 | 5.41333 | 323 | 18.00% |
| 9 | 17.013 | 5.20757 | 1340 | 75.00% |
| 10 | 18.706 | 4.7397 | 570 | 31.90% |
| 11 | 18.966 | 4.67532 | 824 | 46.10% |
| 12 | 19.325 | 4.58943 | 337 | 18.90% |
| 13 | 19.697 | 4.50361 | 710 | 39.70% |
| 14 | 21.761 | 4.08076 | 253 | 14.10% |
| 15 | 22.913 | 3.87816 | 901 | 50.40% |
| 16 | 23.194 | 3.83185 | 359 | 20.10% |
| 17 | 23.696 | 3.75179 | 661 | 37.00% |
| 18 | 26.082 | 3.4137 | 452 | 25.30% |

wherein the XRPD pattern is measured in a diffractometer using copper anode, at wavelength alpha1 of 1.5406 Å and wavelength alpha2 of 1.5444 Å.
1.40 Any of Co-crystals 1.36-1.39, wherein said co-crystal exhibits an X-ray powder diffraction pattern comprising at least five peaks having d-spacing values selected from those set forth in the table of Co-crystal 1.39.
1.41 Any of Co-crystals 1.36-1.40, wherein the co-crystal exhibits an X-ray powder diffraction pattern comprising at least five peaks having d-spacing values selected from the group consisting of 12.14, 12.00, 8.88, 6.27, 5.21, 4.74, 4.68, 4.50, 3.88, and 3.75 Å.
1.42 Any of Co-crystals 1.36-1.41, wherein the co-crystal exhibits an X-ray powder diffraction pattern comprising peaks having d-spacing values of 12.14, 12.00, 5.21, 4.68, and 3.88 Å.
1.43 Any of Co-crystals 1.36-1.42, wherein said co-crystal exhibits an X-ray powder diffraction pattern corresponding with or substantially as depicted in the table of Co-crystal 1.39 or in FIG. 6.
1.44 Any of Co-crystals 1.36-1.43, wherein said co-crystal exhibits a Differential Scanning calorimetry (DSC) pattern comprising an endothermic peak at about 160° C.-162° C., e.g., about 161° C., and about 167° C.-168° C., e.g., about 167.4° C.
1.45 Any of Co-crystals 1.36-1.44, wherein the co-crystal exhibits a Differential Scanning calorimetry (DSC) pattern corresponding with or substantially as depicted in FIG. 7.
1.46 Any of Co-crystals 1.36-1.45, wherein the co-crystal has a needle shape, e.g., having a needle shape with a length of 20-100 μm.
1.47 Any of Co-crystals 1.36-1.46, wherein the co-crystal comprises Compound 1 in free base form and co-crystal former in a ratio of 2:1.
1.48 Any of Co-crystals 1.1-1.9, wherein the co-crystal former is hydroquinone.
1.49 Co-crystals 1.48, wherein the co-crystal exhibits an X-ray powder diffraction pattern comprising at least five peaks having 2-theta angle values selected from the group consisting of: 7.26, 7.42, 9.94, 17.21, 18.41, 18.38, 19.44, 19.47, 23.08, and 23.73 degrees, wherein the XRPD pattern is measured in a diffractometer using copper anode, e.g., at wavelength alpha1 of 1.5406 Å and wavelength alpha2 of 1.5444 Å.
1.50 Any of Co-crystals 1.48-1.49, wherein the co-crystal exhibits an X-ray powder diffraction pattern comprising peaks having 2-theta angle values at 7.26, 7.42, 9.94, 17.21, and 23.08 degrees, wherein the XRPD pattern is measured in a diffractometer using copper anode, e.g., at wavelength alpha1 of 1.5406 Å and wavelength alpha2 of 1.5444 Å.
1.51 Any of Co-crystals 1.48-1.50, wherein the co-crystal exhibits an X-ray powder diffraction pattern comprising at least five peaks having 2-theta angle values selected from those set forth in the table below:

| | | | | Rel. |
|---|---|---|---|---|
| Index | Angle | d Value | Intensity | Intensity |
| 1 | 4.978 | 17.73707 | 885 | 45.70% |
| 2 | 7.262 | 12.16263 | 1470 | 75.90% |
| 3 | 7.419 | 11.90583 | 1930 | 100.00% |
| 4 | 9.941 | 8.8902 | 1250 | 64.80% |
| 5 | 10.276 | 8.60155 | 703 | 36.40% |
| 6 | 13.912 | 6.36035 | 348 | 18.00% |
| 7 | 14.332 | 6.17516 | 467 | 24.10% |
| 8 | 14.787 | 5.98601 | 631 | 32.70% |
| 9 | 15.524 | 5.70357 | 745 | 38.60% |
| 10 | 15.971 | 5.54483 | 654 | 33.80% |
| 11 | 16.655 | 5.31858 | 746 | 38.60% |
| 12 | 17.212 | 5.1477 | 960 | 49.60% |
| 13 | 18.376 | 4.82411 | 891 | 46.10% |
| 14 | 18.406 | 4.81644 | 919 | 47.50% |
| 15 | 18.996 | 4.66814 | 698 | 36.10% |
| 16 | 19.435 | 4.56362 | 911 | 47.10% |
| 17 | 19.465 | 4.55658 | 935 | 48.30% |
| 18 | 23.077 | 3.85093 | 1170 | 60.70% |
| 19 | 23.725 | 3.74728 | 948 | 49.00% |
| 20 | 25.871 | 3.44103 | 593 | 30.70% |

wherein the XRPD pattern is measured in a diffractometer using copper anode, at wavelength alpha1 of 1.5406 Å and wavelength alpha2 of 1.5444 Å.
1.52 Any of Co-crystals 1.48-1.51, wherein said co-crystals exhibits an X-ray powder diffraction pattern comprising at least five peaks having d-spacing values selected from those set forth in the table of Co-crystal 1.51.
1.53 Any of Co-crystals 1.48-1.52, wherein the co-crystal exhibits an X-ray powder diffraction pattern comprising at least five peaks having d-spacing values selected from the group consisting of 17.74, 12.16, 11.91, 8.89, 5.32, 5.15, 4.82, 4.56, 3.85, and 3.75 Å.
1.54 Any of Co-crystals 1.48-1.53, wherein the co-crystal exhibits an X-ray powder diffraction pattern comprising peaks having d-spacing values of 12.16, 11.91, 8.89, 5.15, and 3.85 Å.
1.55 Any of Co-crystals 1.48-1.54, wherein said co-crystal exhibits an X-ray powder diffraction pattern corresponding with or substantially as depicted in the table of Co-crystal 1.51 or in FIG. 8.
1.56 Any of Co-crystals 1.48-1.55, wherein said co-crystal exhibits a Differential Scanning calorimetry (DSC) pattern comprising an endothermic peak at about 209° C.-210° C., e.g., about 209.2° C.
1.57 Any of Co-crystals 1.48-1.56, wherein the co-crystal exhibits a Differential Scanning calorimetry (DSC) pattern corresponding with or substantially as depicted in FIG. 9.
1.58 Any of Co-Crystals 1.48-1.57, wherein said co-crystal exhibits a Differential Scanning calorimetry (DSC) pattern comprising endothermic peaks at about 128° C.-129° C., e.g., about 128.6° C.; about 148° C.-150° C., e.g., about 149° C.; and about 200° C.-201° C., e.g., 200.4° C.
1.59 Any of Co-crystals 1.48-1.58, wherein the co-crystal exhibits a Differential Scanning calorimetry (DSC) pattern corresponding with or substantially as depicted in FIG. 10.
1.60 Any of Co-crystals 1.48-1.59, wherein the co-crystal has a needle shape, e.g., having a needle shape with a length of 2-30 μm.
1.61 Any of Co-crystals 1.48-1.60, wherein the co-crystal comprises Compound 1 in free base form and co-crystal former in a ratio of 2:1.
1.62 Any of the preceding Co-crystals, wherein said Co-crystals are in a single crystal form and are free or substantially free of any other form, e.g., less than 10 wt. %, preferably less than about 5 wt. %, more preferably less than about 2wt. %, still preferably less than about 1 wt. %, still preferably less than about 0.1%, most preferably less than about 0.01 wt. %, of amorphous form.
1.63 Any of the preceding Co-crystals, wherein said Co-crystals are in a single crystal form and are free or substantially free of any other form, e.g., less than 10 wt. %, preferably less than about 5 wt. %, more preferably less than about 2wt. %, still preferably less than about 1 wt. %, still preferably less than about 0.1%, most preferably less than about 0.01 wt. %, of other crystal forms.
1.64 Any of the preceding Co-crystals, wherein said Co-crystals are in a single crystal form and are free or substantially free of any other form, e.g., less than 10 wt. %, preferably less than about 5 wt. %, more preferably less than about 2wt. %, still preferably less than about 1 wt. %, still preferably less than about 0.1%, most preferably less than about 0.01 wt. %, of amorphous and other crystal forms.
1.65 Any of the preceding Co-crystals, wherein said Co-crystal is formed according to Method 1, et seq.

In a further aspect, the present disclosure also provides a process [Method 1] for the production of a co-crystal comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) in free, pharmaceutically acceptable salt or prodrug form, including its enantiomers, diastereoisomers and racemates; and a co-crystal former, the method comprising the steps of reacting Compound 1 with the co-crystal former and isolating the obtained co-crystal.

In various embodiments, the disclosure provides the following:
1.1 Method 1, wherein Compound 1 is in free base or salt form.
1.2 Method 1 or 1.1, wherein Compound 1 is in free base form.
1.3 Any of the preceding Methods, wherein the co-crystal is anhydrous or solvated.
1.4 Any of the preceding Methods, wherein the co-crystal is anhydrous.
1.5 Any of the preceding Methods, wherein the co-crystal is solvated.
1.6 Any of the preceding Methods, wherein the ratio of Compound 1 to co-crystal former in the resulting co-crystal is between 1:0.5 and 1:3 (e.g., 1:0.5, 1:0.7, 1:1 or 1:2).
1.7 Any of the preceding methods, further comprising the step of dissolving Compound 1 in a first solvent comprising an alcohol and/or water.
1.8 Any of the preceding methods, further comprising the step of dissolving Compound 1 in a solvent selected from ethanol, methanol, water, and combinations thereof.
1.9 Any of the preceding methods, further comprising the step of dissolving Compound 1 in a solvent comprising methanol and water.
1.10 The preceding Method, wherein the methanol and water are in a molar ratio between 5:1 and 15:1.
1.11 The preceding Method, wherein the methanol and water are in a molar ratio of 9:1.
1.12 Any of the preceding methods, further comprising the step of dissolving the co-crystal former in a second solvent.
1.13 The preceding method, wherein the second solvent is an alcohol or water.
1.14 Any of the preceding methods, further comprising the step of dissolving the co-crystal former in a second solvent comprising ethanol, methanol or water.
1.15 The preceding method, wherein the second solvent is methanol or water.
1.16 Method 1.12, wherein the second solvent is methanol.
1.17 Method 1.12, wherein the second solvent is water.
1.18 Any of methods, 1.12-1.17, further comprising mixing the resultant solution comprising Compound 1 in the first solvent with the resultant solution comprising the co-crystal former and the second solvent.
1.19 Any of the preceding Methods, wherein the co-crystal former is selected from alanine, glutamic acid, 2-aminobutyric acid, urea, tyrosine, glycine, arginine, 6-hydroxy nicotinamide, diethanolamine, 3-Nitro phthalimide, isoleucine, histidine, bis acetyled ethylenediamide, nicotinamide, acetanilide, leucine, lysine, isonicotinamide, resorcinol, 4-nitro phthalimide, proline, serine, pyridino phthalimide, 4-acetamidophenol Benzamide, valine, threonine, tromethamine, hydroquinone, carbamazepine, phenylalanine, cysteine, 3-aminobutyric acid, piperazine, 4-acetamidophenol, tryptophan, methionine, 6-methylpyridine-3-carboxamide, succinimide, aspartic acid, asparagine, mono acetyled ethylenediamine, 4-methylacetanilide, glutamine, 2-pyridone, pyromellitic diimide, 1,2-dihydroxybenzene, amino-4,6-dimethyl nicotinamide, and salts thereof.
1.20 Any of the preceding Methods, wherein the co-crystal former is selected from tyrosine, 3-nitro-phthalimide, pyridino phthalimide, diethanolamine, resorcinol, hydroquinone, threonine, and salts thereof.
1.21 Any of the preceding Methods, wherein the co-crystal former is selected from 3-nitro-phthalimide, diethanolamine, resorcinol, hydroquinone, and salts thereof.
1.22 Any of the preceding Methods, wherein Compound 1 is in free base form and the co-crystal former is 3-nitro-phthalimide.
1.23 The preceding Method, further comprising dissolving Compound 1 in a mixture of methanol and water in a molar ratio of 9:1, dissolving the 3-nitro-phthalimide in methanol, and mixing the two resultant solutions to form a reaction mixture.
1.24 Any of the preceding Methods, wherein Compound 1 is in free base form and the co-crystal former is diethanolamine.
1.25 The preceding Method, further comprising dissolving Compound 1 in a mixture of methanol and water in a molar ratio of 9:1, dissolving the diethanolamine in water, and mixing the two resultant solutions to form a reaction mixture.
1.26 Any of the preceding Methods, wherein Compound 1 is in free base form and the co-crystal former is resorcinol.
1.27 The preceding Method, further comprising dissolving Compound 1 in a mixture of methanol and water in a molar ratio of 9:1, dissolving the resorcinol in water, and mixing the two resultant solutions to form a reaction mixture.
1.28 Any of the preceding Methods, wherein Compound 1 is in free base form and the co-crystal former is hydroquinone.
1.29 The preceding Method, further comprising dissolving Compound 1 in a mixture of methanol and water in a molar ratio of 9:1, dissolving hydroquinone in water, and mixing the two resultant solutions to form a reaction mixture.
1.30 Any of the preceding methods, wherein Compound 1 and the co-crystal former are reacted in a molar ratio of 2:1 to 1:2.
1.31 The preceding method, wherein Compound 1 and the co-crystal former are reacted in a molar ratio of 1:1.
1.32 Any of the preceding methods, further comprising the step of heating the reaction mixture containing Compound 1 and the co-crystal former, e.g., to a temperature between 30° C.-80° C., e.g., to a temperature of about 50° C.
1.33 Any of the preceding methods, further comprising the step of drying the reaction mixture, e.g., to a temperature between 0° C.-20° C., e.g., a temperature of about 5° C.
1.34 Any of the preceding methods, wherein the co-crystal is a co-crystal according Co-crystal 1, et seq.

The present disclosure further provides a method [Method 2] for the prophylaxis or treatment of a patient, e.g., a human, suffering from a disorder selected from the following disorders:
A. Neurodegenerative diseases, including Parkinson's disease, restless leg, tremors, dyskinesias, Huntington's disease, Alzheimer's disease, and drug-induced movement disorders;
B. Mental disorders, including depression, attention deficit disorder, attention deficit hyperactivity disorder, bipolar illness, anxiety, sleep disorders, e.g., narcolepsy, cognitive impairment, e.g., cognitive impairment of schizophrenia, dementia, Tourette's syndrome, autism, fragile X syndrome, psychostimulant withdrawal, and drug addiction;
C. Circulatory and cardiovascular disorders, including cerebrovascular disease, stroke, congestive heart disease, hypertension, pulmonary hypertension, e.g., pulmonary arterial hypertension, and sexual dysfunction, including cardiovascular diseases and related disorders as described in International Application No. PCT/US2014/16741, the contents of which are incorporated herein by reference;
D. Respiratory and inflammatory disorders, including asthma, chronic obstructive pulmonary disease, and allergic rhinitis, as well as autoimmune and inflammatory diseases;
E. Diseases that may be alleviated by the enhancement of progesterone-signaling such as female sexual dysfunction;
F. A disease or disorder such as psychosis, glaucoma, or elevated intraocular pressure;
G. Traumatic brain injury;
H. Cancers or tumors, e.g., brain tumors, a glioma (e.g., ependymoma, astrocytoma, oligodendrogliomas, brain stem glioma, optic nerve glioma, or mixed gliomas, e.g., oligoastrocytomas), an astrocytoma (e.g., glioblastoma multiforme), osteosarcoma, melanoma, leukemia, neuroblastoma or leukemia;
I. Renal disorders, e.g., kidney fibrosis, chronic kidney disease, renal failure, glomerulosclerosis and nephritis;
J. Any disease or condition characterized by low levels of cAMP and/or cGMP (or inhibition of cAMP and/or cGMP signaling pathways) in cells expressing PDE1; and/or
K. Any disease or condition characterized by reduced dopamine D1 receptor signaling activity,
   comprising administering to a patient in need thereof a therapeutically effective amount of a co-crystal [Co-crystal 1] comprising:
i. (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) in free, pharmaceutically acceptable salt or prodrug form, including its enantiomers, diastereoisomers and racemates; and
ii. a co-crystal former,
   e.g., Co-crystal 1 et seq., of the present disclosure.
      2.1 A pharmaceutical composition comprising Co-crystal 1 et seq. for use as a medicament, e.g., for use in the manufacture of a medicament for the treatment or prophylaxis of a disease as described in Method 2.
      2.2 Use of Co-crystal 1 et seq. in the treatment or prophylaxis of a disease as described in Method 2.
      2.3 Method 2, wherein the co-crystal is administered to a patient in an amount equivalent to 1-300 mg of Compound 1 per day.
      2.4 Any of the preceding methods, wherein the co-crystal is administered to a patient in an amount equivalent to 15-180 mg per day, e.g., 30-90 mg per day, e.g., 30-60 mg of Compound 1 per day.

### BRIEF DESCRIPTION OF THE DRAWINGS

FIG. 1 depicts an x-ray powder diffraction pattern of a (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1)-3-nitro-phthalimide co-crystal.

FIG. 2 depicts a differential scanning calorimetry (DSC) thermograph of a Compound 1-3-nitro-phthalimide co-crystal.

FIG. 3 depicts a differential scanning calorimetry (DSC) thermograph of a Compound 1-3-nitro-phthalimide co-crystal.

FIG. 4 depicts an x-ray powder diffraction pattern of a Compound 1-diethanolamine co-crystal.

FIG. 5 depicts a differential scanning calorimetry (DSC) thermograph pattern of the Compound 1-diethanolamine co-crystal.

FIG. 6 depicts an x-ray powder diffraction pattern of a Compound 1-resorcinol co-crystal.

FIG. 7 depicts a differential scanning calorimetry (DSC) thermograph pattern of the Compound 1-resorcinol co-crystal.

FIG. 8 depicts an x-ray powder diffraction pattern of a Compound 1-hydroquinone co-crystal.

FIG. 9 depicts a differential scanning calorimetry (DSC) thermograph of a Compound 1-hydroquinone co-crystal.

FIG. 10 depicts a differential scanning calorimetry (DSC) thermograph of a Compound 1-hydroquinone co-crystal.

### DETAILED DESCRIPTION

As use herein, the term “crystal” or “crystals” or “crystalline” or “crystallinic” refers to any solid that has a short-or long-range order of the molecules, atoms or ions in a fixed lattice arrangement. The co-crystals of the present disclosure may be in a single crystal form. Therefore, the co-crystals of the present disclosure may be in a triclinic, monoclinic, orthorhombic, tetragonal, rhobohedral, hexagonal or cubic crystal form or mixtures thereof. In particular, the co-crystals of the present disclosure are in dry crystalline form. In another embodiment, the co-crystals of the present disclosure are in needle form. In a particular embodiment, the co-crystals of the present disclosure are substantially free of other forms, e.g., free of amorphous or other crystal forms.

The term “substantially free” of other crystal forms refer to less than about 10 wt. %, preferably less than about 5 wt. %, more preferably less than about 2 wt. %, still preferably less than about 1 wt. %, still preferably less than about 0.1%, most preferably less than about 0.01 wt. % of other forms or other crystal forms, e.g., amorphous or other crystal forms.

In a particular embodiment, the crystals of the disclosure may contain trace amounts of solvent, e.g., in solvate form, or trace amounts of water, e.g., in hydrate form. The co-crystals of the present disclosure are in non-solvate form. In some embodiments, the crystals of the disclosure are in non-solvate and non-hydrate form. In some embodiments, the co-crystals of the present disclosure are in anhydrous form.

The co-crystals of the present disclosure may have a Compound 1 to co-crystal former ratio of between about 5:1 and 1:5. For example, in various embodiments, the ratio of Compound 1 to co-crystal former may be between 1:2 and 2:1, e.g., 1:1, 1:0.5, 1:0.7 or 1:>1, e.g., 1:1.3 or 1:2, etc. For example, the Compound 1-3-nitro-phthalimide co-crystal of the disclosure may comprise Compound 1 and 3-nitro-phthalimide in a ratio of 1:0.5 and/or 1:1 (e.g., 1:0.7). The Compound 1-diethanolamine co-crystal of the disclosure may comprise Compound 1 and diethanolamine in a ratio of 1:1. The Compound 1-resorcinol co-crystal of the disclosure may comprise Compound 1 and resorcinol in a ratio of 2:1. The Compound 1-hydroquinone co-crystal of the disclosure may comprise Compound 1 and hydroquinone in a ratio of 2:1.

The term “solvate” refers to crystalline solid adducts containing either stoichiometric or nonstoichiometric amounts of a solvent incorporated within the crystal structure. Therefore, the term “non-solvate” form herein refers to co-crystals that are free or substantially free of solvent molecules within the crystal structures of the disclosure. Similarly, the term “non-hydrate” form herein refers to co-crystals that are free or substantially free of water molecules within the crystal structures of the disclosure.

The term “amorphous” form refers to solids of disordered arrangements of molecules and do not possess a distinguishable crystal lattice.

Unless further modified, the term “Compound 1” refers to (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one in free base form, having the following structure:

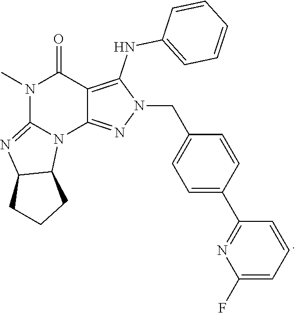 [SMILES: CN1C(=O)c2c(nn(Cc3ccc(-c4cccc(F)n4)cc3)c2Nc2ccccc2)N2C1=N[C@@H]1CCC[C@@H]12]

The crystallinity or the morphology of the co-crystals of the present disclosure may be determined by a number of methods, including, but not limited to single crystal X-ray diffraction, X-ray powder diffraction, polarizing optical microscopy, thermal microscopy, differential scanning calorimetry (DSC), thermogravimetric analysis (TGA), infrared adsorption spectroscopy and Raman spectroscopy. Characterization of solvates or hydrates or lack thereof may also be determined by DSC and/or TGA.

It is to be understood that X-ray powder diffraction pattern or the differential scanning calorimetry pattern of a given sample may vary a little (standard deviation) depending on the instrument used, the time and temperature of the sample when measured and standard experimental errors. Therefore, the temperature or the 2-theta values, d-spacing values, heights and relative intensity of the peaks as set forth herein in the Tables or in the Figures will have an acceptable level of deviation. For example, the values may have an acceptable deviation of e.g., about 20%, 15%, 10%, 5%, 3%, 2% or 1%. In particular embodiment, the 2-theta values or the d-spacing values of the XRPD pattern of the crystals of the current disclosure may have an acceptable deviation of ±0.2 degrees and/or ±0.2 Å. Further, the XRPD pattern of the crystals of the disclosure may be identified by the characteristic peaks as recognized by one skilled in the art. For example, the crystals of the disclosure may be identified by e.g., at least five characteristic peaks, e.g., at least three or at least five peaks, e.g., at least three or at least five 2-theta values and/or at least three or at least five d-spacing values as set forth in the XRPD patterns set forth herein. Therefore, the term “corresponding with or substantially as” set forth in any of the Tables or depicted in any of the Figures refers to any crystals which has an XRPD having the major or characteristic peaks as set forth in the tables/figures.

The term “about” in front of a numerical value refers to the numerical value itself ±20%, ±15%, ±10%, preferably ±5%, preferably ±3%, preferably ±2%, preferably ±1% of that value. When referencing temperature, the term about refers to the temperature value itself ±10° C., preferably ±5° C., preferably ±3° C. of the reference temperature. In another example, when referencing 2-theta angle values, the term “about” refers to the numerical 2-theta angle value itself ±0.2 degrees of the reference 2-theta angle value. In still another example, when referencing d-spacing values, the term “about” refers to the numerical 2-theta angle value itself ±0.2 Å of the reference d-spacing value.

The crystals of the disclosure are selective PDE1 inhibitors. Therefore, the crystals of the disclosure are useful for the treatment of PDE1 related disorders as set forth in e.g., WO 2014/151409, WO 2018/049417, WO 2019/227004, WO 2019/152697, WO 2009/075784, WO 2010/132127, WO 2006/133261 and WO 2011/153129, the contents of each of which are incorporated by reference in their entireties.

The term “patient” includes human and non-human. In one embodiment, the patient is a human. In another embodiment, the patient is a non-human.

### EXAMPLES

### Example 1: Free Base Crystals

The method of making the compound (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one is generally described in WO 2009/075784, the contents of which are incorporated by reference in its entirety. This compound can also be prepared as summarized or similarly summarized in the following reaction Schemes 1 and 2.

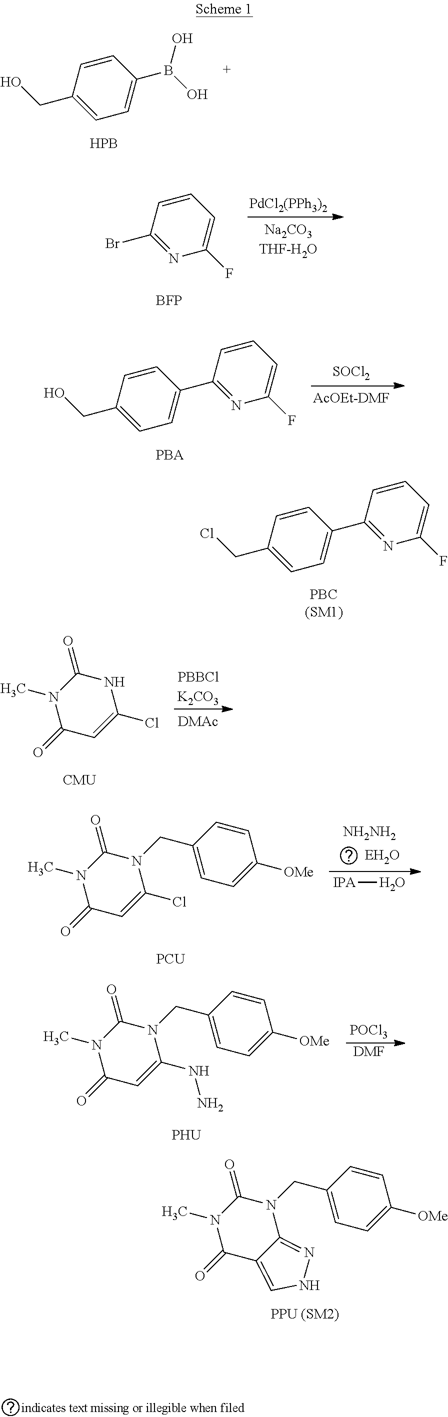

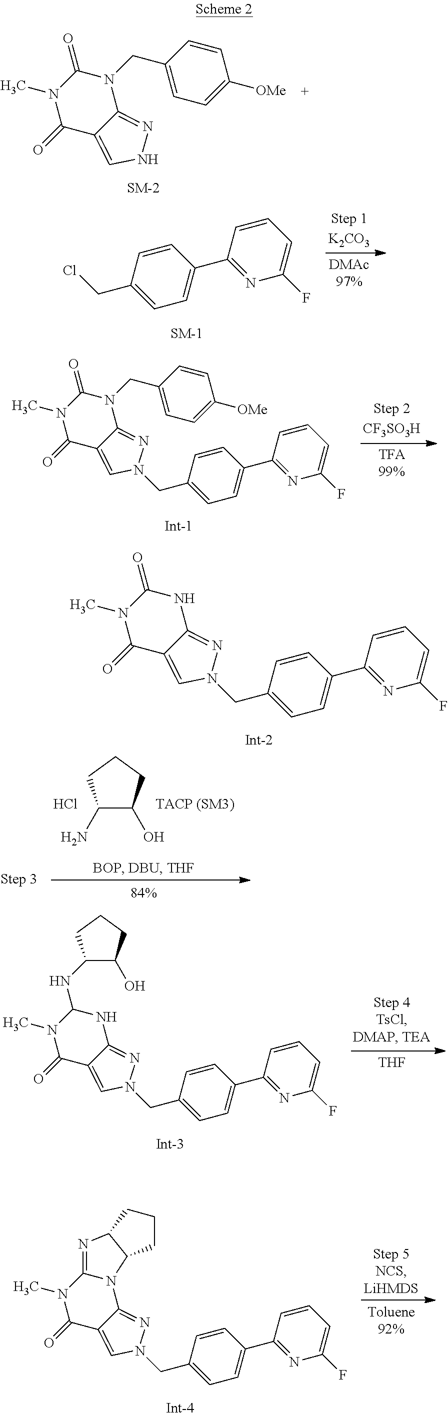

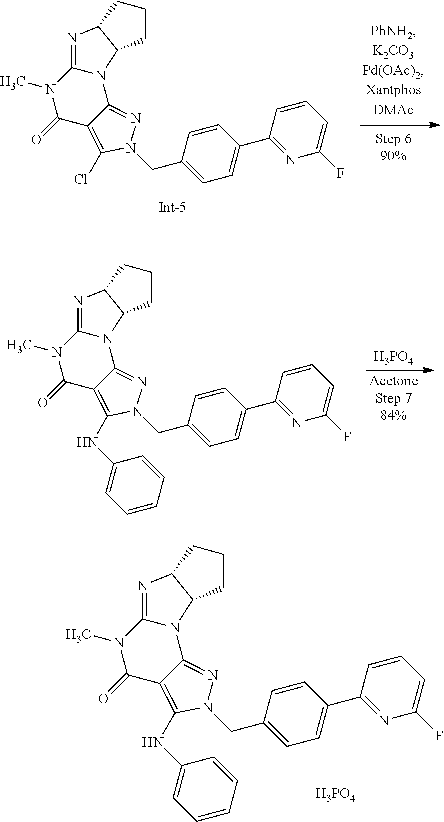

Crystalline (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) free base may be created according to the methods as described in, for example, WO2014205354 A1, the contents of which are incorporated by reference in its entirety.

### Example 2:3-nitro-phthalimide Co-Crystal

100 mg of Compound 1 free base is added to a vial and dissolved in a 9:1 mixture of methanol and water (2000 μl). The 3-nitro-phthalimide co-former (molar ratio 1:1 to Compound 1 Free Base) is dissolved in methanol, and added to the Free Base solution. The vial was then shaken at 50° C. for three hours. The obtained clear liquid is transferred to a cooling plate which was cooled to 5° C. and stored at this temperature for about 16 hours. The remaining liquids are absorbed by filter paper and stored in vacuum until completely dry.

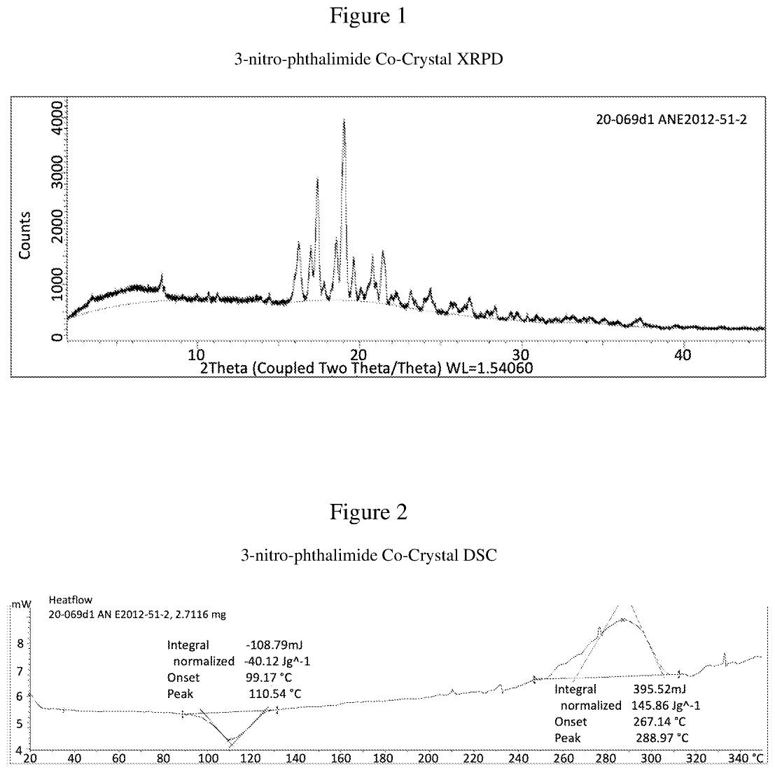

The obtained solid is dried in vacuum and characterized first through XRPD. The X-ray powder diffraction studies are performed using a Bruker AXS D2 PHASER in Bragg-Brentano configuration. The X-ray source is a Cu anode at 30 kV, 10 mA. The resulting XRPD is shown in FIG. 1, and is summarized below in Table 1. The observed crystals are solvated as a methanol solvate and needle-shaped, having a size of about 50-200 μm.

TABLE 1

| | | | | Rel. |
|---|---|---|---|---|
| Index | Angle | d Value | Intensity | Intensity |
| 1 | 7.807 | 11.31578 | 467 | 14.50% |
| 2 | 10.695 | 8.2655 | 173 | 5.40% |
| 3 | 16.256 | 5.4483 | 1010 | 31.50% |
| 4 | 16.989 | 5.2148 | 953 | 29.60% |
| 5 | 17.403 | 5.09173 | 2210 | 68.80% |
| 6 | 17.817 | 4.97425 | 341 | 10.60% |
| 7 | 18.571 | 4.77403 | 1110 | 34.50% |
| 8 | 19.052 | 4.65455 | 3220 | 100.00% |
| 9 | 19.637 | 4.51712 | 743 | 23.10% |
| 10 | 20.806 | 4.26583 | 838 | 26.00% |
| 11 | 21.026 | 4.22184 | 379 | 11.80% |
| 12 | 21.431 | 4.14283 | 929 | 28.90% |
| 13 | 21.531 | 4.12395 | 821 | 25.50% |
| 14 | 23.171 | 3.83555 | 274 | 8.50% |
| 15 | 24.355 | 3.65167 | 397 | 12.30% |

The XRPD data shows that a crystalline structure was formed. Differential Scanning calorimetry is carried out on a Mettler Toledo TGA/DSC-3+ STARe System with a 34-position auto sampler. 5-10 mg of sample is loaded into a pre-weighed Aluminum crucible and is kept at 20° C. for 5 minutes, after which it is heated at 10° C./min from 20° C. to 350° C. A nitrogen purge of 40 ml/min is maintained over the sample. Readings are taken using STARe Software v15.00 build 8668. The DSC curve obtained is illustrated in FIG. 2, which shows an endothermic peak at 111° C.

Co-crystal formation was confirmed via 1H-NMR and FT-IR.

### Example 3:3-nitro-phthalimide Co-Crystal

20 mg of Compound 1 free base is added to a vial and dissolved in a 9:1 mixture of methanol and water (400 μl). The 3-nitro-phthalimide co-former (molar ratio 1:1 to Compound 1 Free Base) is dissolved in methanol (400 μl), and added to the Free Base solution. The vial is then shaken at 50° C. for three hours. The obtained clear liquid is transferred to a cooling plate which is cooled to 5° C. and stored at this temperature for about 16 hours. The remaining liquids are absorbed by filter paper and stored in vacuum until completely dry.

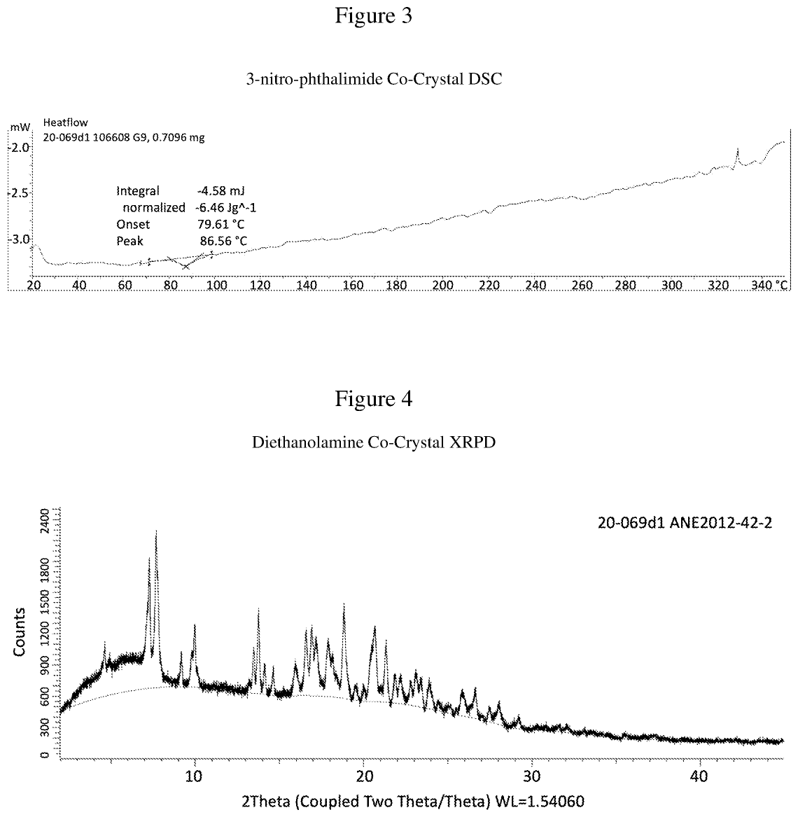

Differential Scanning calorimetry is carried out on a Mettler Toledo TGA/DSC-3+ STARe System with a 34-position auto sampler. 5-10 mg of sample is loaded into a pre-weighed Aluminum crucible and is kept at 20° C. for 5 minutes, after which it is heated at 10° C./min from 20° C. to 350° C. A nitrogen purge of 40 ml/min is maintained over the sample. Readings are taken using STARe Software v15.00 build 8668. The DSC curve obtained is illustrated in FIG. 3, which shows an endothermic peak at 87° C.

### Example 4: Diethanolamine Co-Crystal

100 mg of Compound 1 free base is added to a vial and dissolved in a 9:1 mixture of methanol and water (2000 μl). The diethanolamine co-former (molar ratio 1:1 to Compound 1 Free Base) is dissolved in water, and added to the Free Base solution. The vial is then shaken at 50° C. for three hours. The obtained clear liquid is transferred to a cooling plate which was cooled to 5° C. and stored at this temperature for about 16 hours. The remaining liquids are absorbed by filter paper and stored in vacuum until completely dry.

The obtained solid is dried in vacuum and characterized first through XRPD. The X-ray powder diffraction studies are performed using a Bruker AXS D2 PHASER in Bragg-Brentano configuration. The X-ray source is a Cu anode at 30 kV, 10 mA. The resulting XRPD is shown in FIG. 4, and is summarized below in Table 2. The observed crystals are anhydrous and needle-shaped, having a size of about 5-40 μm.

TABLE 2

| | | | | Rel. |
|---|---|---|---|---|
| Index | Angle | d Value | Intensity | Intensity |
| 1 | 4.636 | 19.04449 | 479 | 32.50% |
| 2 | 7.286 | 12.1232 | 1250 | 84.90% |
| 3 | 7.702 | 11.46886 | 1470 | 100.00% |
| 4 | 7.795 | 11.33322 | 1030 | 70.00% |
| 5 | 9.196 | 9.60955 | 314 | 21.30% |
| 6 | 9.987 | 8.85011 | 604 | 40.90% |
| 7 | 13.486 | 6.56056 | 433 | 29.30% |
| 8 | 13.773 | 6.42438 | 810 | 54.90% |
| 9 | 14.123 | 6.26599 | 285 | 19.30% |
| 10 | 14.63 | 6.05003 | 199 | 13.50% |
| 11 | 16.587 | 5.34025 | 625 | 42.40% |
| 12 | 16.931 | 5.23254 | 635 | 43.00% |
| 13 | 17.904 | 4.95021 | 474 | 32.10% |
| 14 | 18.841 | 4.70617 | 910 | 61.70% |
| 15 | 20.39 | 4.35202 | 381 | 25.90% |
| 16 | 20.678 | 4.29208 | 730 | 49.50% |
| 17 | 20.66 | 4.2957 | 693 | 47.00% |
| 18 | 21.338 | 4.16067 | 539 | 36.60% |
| 19 | 23.108 | 3.84586 | 343 | 23.30% |
| 20 | 26.631 | 3.34459 | 281 | 19.00% |

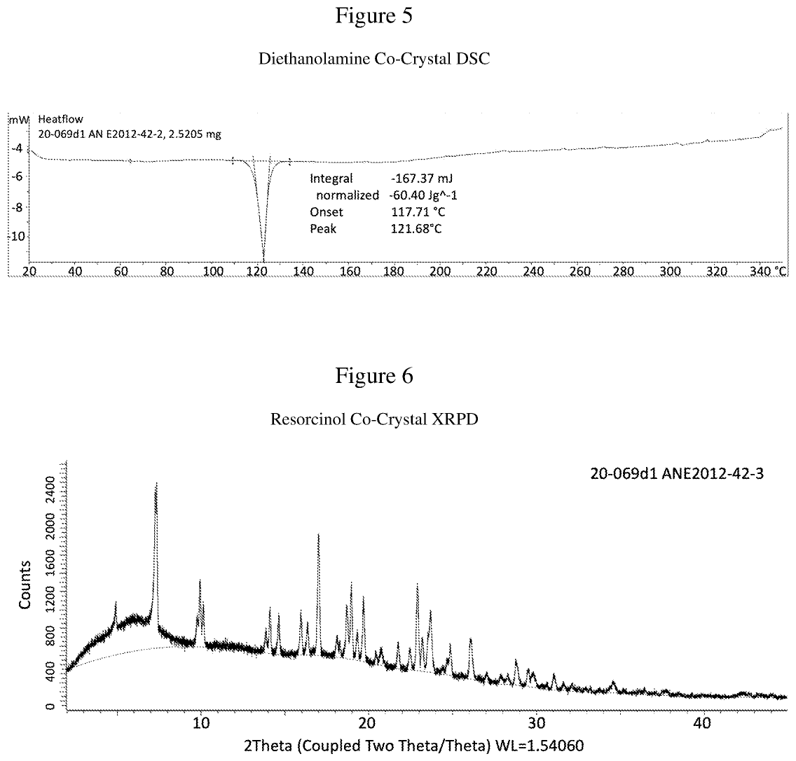

The XRPD data shows that a crystalline structure is formed. Differential Scanning calorimetry is carried out on a Mettler Toledo TGA/DSC-3+ STARe System with a 34-position auto sampler. 5-10 mg of sample is loaded into a pre-weighed Aluminum crucible and is kept at 20° C. for 5 minutes, after which it is heated at 10° C./min from 20° C. to 350° C. A nitrogen purge of 40 ml/min is maintained over the sample. Readings are taken using STARe Software v15.00 build 8668. The DSC curve obtained is illustrated in FIG. 5, which shows an endothermic peak at 122° C.

Co-crystal formation is confirmed via 1H-NMR and FT-IR. 1H-NMR shows that the co-crystal includes the free base and co-former in a ratio of 1:1.

### Example 5: Resorcinol Co-Crystal

100 mg of Compound 1 free base is added to a vial and dissolved in a 9:1 mixture of methanol and water (2000 μl). The resorcinol co-former (molar ratio 1:1 to Compound 1 Free Base) is dissolved in water, and added to the Free Base solution. The vial is then shaken at 50° C. for three hours. The obtained clear liquid is transferred to a cooling plate which was cooled to 5° C. and stored at this temperature for about 16 hours. The remaining liquids are absorbed by filter paper and stored in vacuum until completely dry.

The obtained solid is dried in vacuum and characterized first through XRPD. The X-ray powder diffraction studies are performed using a Bruker AXS D2 PHASER in Bragg-Brentano configuration. The X-ray source is a Cu anode at 30 kV, 10 mA. The resulting XRPD is shown in FIG. 6, and is summarized below in Table 3. The observed crystals are anhydrous and needle- shaped, having a size of about 20-100 μm.

TABLE 3

| | | | | Rel. |
|---|---|---|---|---|
| Index | Angle | d Value | Intensity | Intensity |
| 1 | 7.276 | 12.13973 | 1760 | 98.40% |
| 2 | 7.36 | 12.00098 | 1790 | 100.00% |
| 3 | 9.952 | 8.88113 | 737 | 41.20% |
| 4 | 10.146 | 8.7117 | 465 | 26.00% |
| 5 | 14.118 | 6.26829 | 511 | 28.60% |
| 6 | 14.649 | 6.04204 | 397 | 22.20% |
| 7 | 15.962 | 5.54788 | 478 | 26.70% |
| 8 | 16.362 | 5.41333 | 323 | 18.00% |
| 9 | 17.013 | 5.20757 | 1340 | 75.00% |
| 10 | 18.706 | 4.7397 | 570 | 31.90% |
| 11 | 18.966 | 4.67532 | 824 | 46.10% |
| 12 | 19.325 | 4.58943 | 337 | 18.90% |
| 13 | 19.697 | 4.50361 | 710 | 39.70% |
| 14 | 21.761 | 4.08076 | 253 | 14.10% |
| 15 | 22.913 | 3.87816 | 901 | 50.40% |
| 16 | 23.194 | 3.83185 | 359 | 20.10% |
| 17 | 23.696 | 3.75179 | 661 | 37.00% |
| 18 | 26.082 | 3.4137 | 452 | 25.30% |

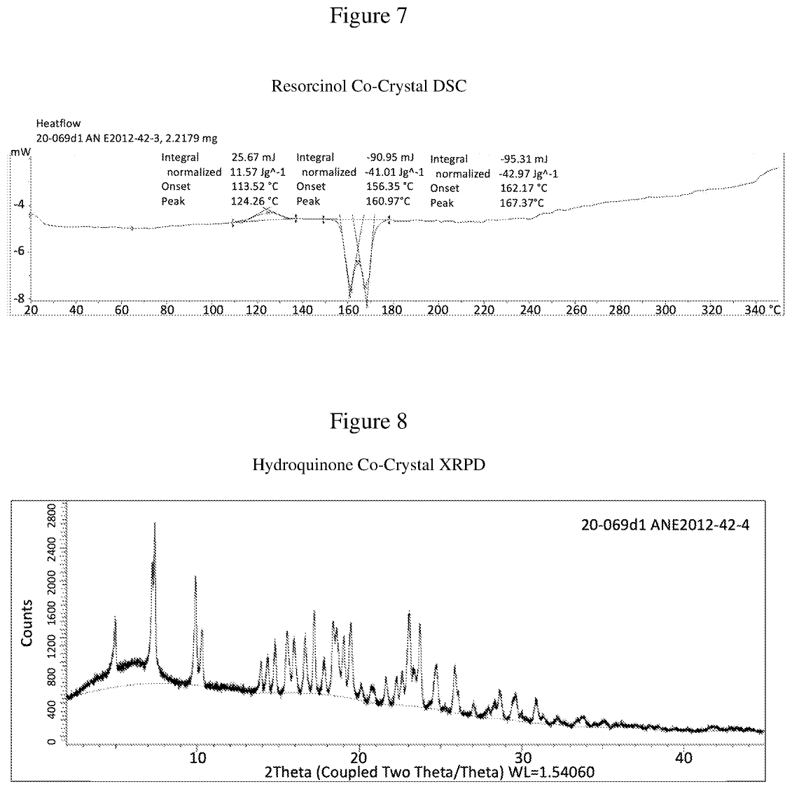

The XRPD data shows that a crystalline structure is formed. Differential Scanning calorimetry is carried out on a Mettler Toledo TGA/DSC-3+ STARe System with a 34-position auto sampler. 5-10 mg of sample is loaded into a pre-weighed Aluminum crucible and is kept at 20° C. for 5 minutes, after which it is heated at 10° C./min from 20° C. to 350° C. A nitrogen purge of 40 ml/min is maintained over the sample. Readings are taken using STARe Software v15.00 build 8668. The DSC curve obtained is illustrated in FIG. 7, which shows endothermic peaks at 161° C. and 167° C.

Co-crystal formation is confirmed via 1H-NMR and FT-IR. 1H-NMR shows that the co-crystal includes the free base and co-former in a ratio of 2:1.

### Example 6: Hydroquinone Co-Crystal

100 mg of Compound 1 free base is added to a vial and dissolved in a 9:1 mixture of methanol and water (2000 μl). The hydroquinone co-former (molar ratio 1:1 to Compound 1 Free Base) is dissolved in water, and added to the Free Base solution. The vial is then shaken at 50° C. for three hours. The obtained clear liquid is transferred to a cooling plate which was cooled to 5° C. and stored at this temperature for about 16 hours. The remaining liquids are absorbed by filter paper and stored in vacuum until completely dry.

The obtained solid is dried in vacuum and characterized first through XRPD. The X-ray powder diffraction studies are performed using a Bruker AXS D2 PHASER in Bragg-Brentano configuration. The X-ray source is a Cu anode at 30 kV, 10 mA. The resulting XRPD is shown in FIG. 8, and is summarized below in Table 4. The observed crystals are anhydrous and needle-shaped, having a size of about 2-30 μm.

TABLE 4

| | | | | Rel. |
|---|---|---|---|---|
| Index | Angle | d Value | Intensity | Intensity |
| 1 | 4.978 | 17.73707 | 885 | 45.70% |
| 2 | 7.262 | 12.16263 | 1470 | 75.90% |
| 3 | 7.419 | 11.90583 | 1930 | 100.00% |
| 4 | 9.941 | 8.8902 | 1250 | 64.80% |
| 5 | 10.276 | 8.60155 | 703 | 36.40% |
| 6 | 13.912 | 6.36035 | 348 | 18.00% |
| 7 | 14.332 | 6.17516 | 467 | 24.10% |
| 8 | 14.787 | 5.98601 | 631 | 32.70% |
| 9 | 15.524 | 5.70357 | 745 | 38.60% |
| 10 | 15.971 | 5.54483 | 654 | 33.80% |
| 11 | 16.655 | 5.31858 | 746 | 38.60% |
| 12 | 17.212 | 5.1477 | 960 | 49.60% |
| 13 | 18.376 | 4.82411 | 891 | 46.10% |
| 14 | 18.406 | 4.81644 | 919 | 47.50% |
| 15 | 18.996 | 4.66814 | 698 | 36.10% |
| 16 | 19.435 | 4.56362 | 911 | 47.10% |
| 17 | 19.465 | 4.55658 | 935 | 48.30% |
| 18 | 23.077 | 3.85093 | 1170 | 60.70% |
| 19 | 23.725 | 3.74728 | 948 | 49.00% |
| 20 | 25.871 | 3.44103 | 593 | 30.70% |

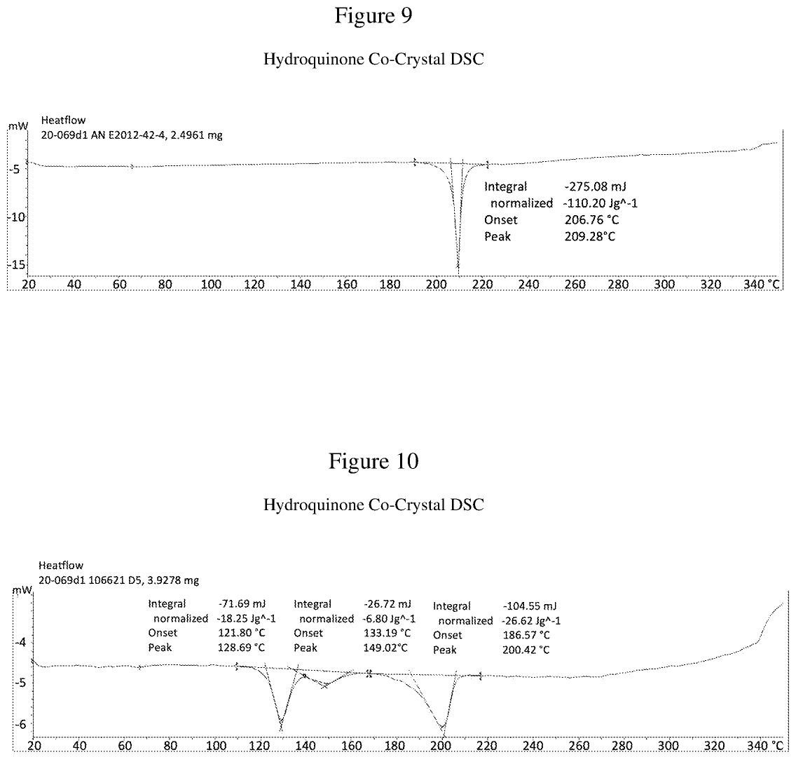

The XRPD data shows that a crystalline structure is formed. Differential Scanning calorimetry is carried out on a Mettler Toledo TGA/DSC-3+ STARe System with a 34-position auto sampler. 5-10 mg of sample is loaded into a pre-weighed Aluminum crucible and is kept at 20° C. for 5 minutes, after which it is heated at 10° C./min from 20° C. to 350° C. A nitrogen purge of 40 ml/min is maintained over the sample. Readings are taken using STARe Software v15.00 build 8668. The DSC curve obtained is illustrated in FIG. 9, which shows an endothermic peak at 208° C.

Co-crystal formation is confirmed via 1H-NMR and FT-IR. 1H-NMR shows that the co-crystal includes the free base and co-former in a ratio of 2:1.

### Example 7: Hydroquinone Co-Crystal

20 mg of Compound 1 free base is added to a vial and dissolved in a 9:1 mixture of methanol and water (400 μl). The hydroquinone co-former (molar ratio 1:1 to Compound 1 Free Base) is dissolved in water (400 μl), and added to the Free Base solution. The vial is then shaken at 50° C. for three hours. The obtained clear liquid is transferred to a cooling plate which is cooled to 5° C. and stored at this temperature for about 16 hours. The remaining liquids are absorbed by filter paper and stored in vacuum until completely dry.

Differential Scanning calorimetry is carried out on a Mettler Toledo TGA/DSC-3+ STARe System with a 34-position auto sampler. 5-10 mg of sample is loaded into a pre-weighed Aluminum crucible and is kept at 20° C. for 5 minutes, after which it is heated at 10° C./min from 20° C. to 350° C. A nitrogen purge of 40 ml/min is maintained over the sample. Readings are taken using STARe Software v15.00 build 8668. The DSC curve obtained is illustrated in FIG. 10, which shows endothermic peaks at 129° C., 149° C., and 200° C.

### Example 8: Co-Crystal Solubility Comparison

The aqueous solubility of the co-crystals is determined by creating saturated solutions containing individual co-crystals in water. The saturated solutions are shaken for 24 hours at room temperature. Samples are taken at two timepoints (2 hours and 24 hours) and are filtered and diluted (acetonitrile/water 1:1) before measurement with liquid chromatography. Results are summarized below in Table 5.

TABLE 5

| | Aqueous solubility | Aqueous solubility |
|---|---|---|
| | after 2 hours | after 24 hours |
| Co-crystal | (mg/mL) | (mg/mL) |
| Free Base - | 0.23 | 1.10 |
| 3-Nitrophthalimide | | |
| Free Base - Diethanolamine | 0 | 0 |
| Free Base - Resorcinol | 0.03 | 0.001 |
| Free Base - Hydroquinone | 0 | 0 |

The solubility of diethanolamine and resorcinol are very low after 24 hours. Hydroquinone is not shown to be soluble at all. Only the Free Base-3-Nitrophthalimide co-crystal showed a solubility of 1.10 mg/mL after 24 hours, which is a significant increase compared to a solubility of 0.001 mg/mL for the Free Base.

1. A co-crystal comprising:
A) (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) in free, pharmaceutically acceptable salt or prodrug form, including its enantiomers, diastereoisomers and racemates; and
B) a co-crystal former.

2. (canceled)

3. The co-crystal according to claim 1, wherein Compound 1 is in free base form.

4. The co-crystal according to claim 1, wherein the co-crystal is anhydrous or solvated.

5. The co-crystal according to claim 1, wherein the co-crystal former is selected from alanine, glutamic acid, 2-aminobutyric acid, urea, tyrosine, glycine, arginine, 6-hydroxy nicotinamide, diethanolamine, 3-nitro-phthalimide, isoleucine, histidine, bis acetyled ethylenediamide, nicotinamide, acetanilide, leucine, lysine, isonicotinamide, resorcinol, 4-nitro phthalimide, proline, serine, pyridino phthalimide, 4-acetamidophenol Benzamide, valine, threonine, tromethamine, hydroquinone, carbamazepine, phenylalanine, cysteine, 3-aminobutyric acid, piperazine, 4-acetamidophenol, tryptophan, methionine, 6-methylpyridine-3-carboxamide, succinimide, aspartic acid, asparagine, mono acetyled ethylenediamine, 4-methylacetanilide, glutamine, 2-pyridone, pyromellitic diimide, 1,2-dihydroxybenzene, amino-4,6-dimethyl nicotinamide, and salts thereof.

6. (canceled)

7. (canceled)

8. The co-crystal according to claim 1, wherein the co-crystal former is 3-nitro-phthalimide.

9. The co-crystal according to claim 8, wherein the co-crystal exhibits an X-ray powder diffraction pattern comprising at least five peaks having 2-theta angle values selected from the group consisting of: 7.8, 16.3, 17.0, 17.4, 18.6, 19.1, 19.6, 20.8, 21.4, and 21.5 degrees, wherein the XRPD pattern is measured in a diffractometer using copper anode, e.g., at wavelength alpha1 of 1.5406 Å and wavelength alpha2 of 1.5444 Å.

10. (canceled)

11. (canceled)

12. (canceled)

13. The co-crystal according to claim 8, wherein said co-crystal exhibits a Differential Scanning calorimetry (DSC) pattern comprising an endothermic peak at about 110° C.-111° C., e.g., about 110.5° C.

14. (canceled)

15. The co-crystal according to claim 1, wherein the co-crystal former is diethanolamine.

16. The co-crystal according to claim 15, wherein the co-crystal exhibits an X-ray powder diffraction pattern comprising at least five peaks having 2-theta angle values selected from the group consisting of: 7.29, 7.70, 7.80, 9.987, 13.78, 16.59, 16.93, 18.84, 20.66, and 20.68 degrees, wherein the XRPD pattern is measured in a diffractometer using copper anode, e.g., at wavelength alpha1 of 1.5406 Å and wavelength alpha2 of 1.5444 Å.

17. (canceled)

18. (canceled)

19. (canceled)

20. The co-crystal according to claim 15, wherein said co-crystal exhibits a Differential Scanning calorimetry (DSC) pattern comprising an endothermic peak at about 121° C.-122° C., e.g., about 121.7° C.

21. The co-crystal according to claim 1, wherein the co-crystal former is resorcinol.

22. The co-crystal according to claim 21, wherein the co-crystal exhibits an X-ray powder diffraction pattern comprising at least five peaks having 2-theta angle values selected from the group consisting of: 7.3, 7.4, 10.0, 14.1, 17.0, 18.7, 19.0, 19.7, 22.9, and 23.7 degrees, wherein the XRPD pattern is measured in a diffractometer using copper anode, e.g., at wavelength alpha1 of 1.5406 Å and wavelength alpha2 of 1.5444 Å.

23. (canceled)

24. (canceled)

25. (canceled)

26. The co-crystal according to claim 21, wherein said co-crystal exhibits a Differential Scanning calorimetry (DSC) pattern comprising an endothermic peak at about 160° C.-162° C., e.g., about 161° C., and about 167° C.-168° C., e.g., about 167.4° C.

27. The co-crystal according to claim 1, wherein the co-crystal former is hydroquinone.

28. The co-crystal according to claim 27, wherein the co-crystal exhibits an X-ray powder diffraction pattern comprising at least five peaks having 2-theta angle values selected from the group consisting of: 7.26, 7.42, 9.94, 17.21, 18.41, 18.38, 19.44, 19.47, 23.08, and 23.73 degrees, wherein the XRPD pattern is measured in a diffractometer using copper anode, e.g., at wavelength alpha1 of 1.5406 Å and wavelength alpha2 of 1.5444 Å.

29. (canceled)

30. (canceled)

31. (canceled)

32. The co-crystal according to claim 27, wherein said co-crystal exhibits a Differential Scanning calorimetry (DSC) pattern comprising an endothermic peak at about 209° C.-210° C., e.g., about 209.2° C.

33. (canceled)

34. A process for the production of a co-crystal comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) in free, pharmaceutically acceptable salt or prodrug form, including its enantiomers, diastereoisomers and racemates; and a co-crystal former, the method comprising the steps of reacting Compound 1 with the co-crystal former and isolating the obtained co-crystal.

35. The process according to claim 34, further comprising the step of dissolving Compound 1 in a first solvent comprising an alcohol and/or water.

36. (canceled)

37. The process according to claim 34, further comprising the step of dissolving the co-crystal former in a second solvent.

38. (canceled)

39. The process according to claim 37, further comprising mixing the resultant solution comprising Compound 1 in the first solvent with the resultant solution comprising the co-crystal former and the second solvent.

40. (canceled)

41. (canceled)

42. (canceled)

43. (canceled)

44. (canceled)
